(19) United States
(12) Patent Application Publication
(10) Pub. No.: US 2006/0129026 A1
(43) Pub. Date: Jun. 15, 2006

(54) Apparatus and method for mounting a cardiac harness on the heart

(76) Inventors: Joshua Wallin, San Jose, CA (US); Christine Palma, Sunnyvale, CA (US)

Correspondence Address: FULWIDER PATTON, 6060 CENTER DRIVE, 10TH FLOOR, LOS ANGELES, CA, 90045, US

(21) Appl. No.: 11/014,145

(22) Filed: Dec. 15, 2004

(51) Int. Cl. A61F 2/00; B28B 1/14

(52) U.S. Cl. 600/37; 264/299

(57) ABSTRACT

A suction cup for use with a delivery device for delivering a cardiac harness to a heart. The suction cup includes a cup body formed of a flexible material, such as silicone, and the cup body has an upper portion and a lower portion, wherein the lower portion defines an opening leading to an inner area of the cup body. The suction cup also includes a connecting aperture formed of a rigid material that is attached to the upper portion of the cup body. The flexible material of the cup body allows the suction cup to rotate relative to the heart tissue, and to re-center itself onto the apex of the heart when the suction cup is improperly aligned with the apex, all while preserving a vacuum attachment to the heart.

### BACKGROUND OF THE INVENTION

1. Field of the Invention

The present invention relates to a device for treating heart failure. More specifically, the invention relates to a suction cup configured to attach to the apex of the heart to counteract the deployment forces required to advance a cardiac harness over the heart.

2. General Background and State of the Art

Congestive heart failure (“CHF”) is characterized by the failure of the heart to pump blood at sufficient flow rates to meet the metabolic demand of tissues, especially the demand for oxygen. It has been determined that a passive wrap, or cardiac harness, may increase the efficiency of a heart affected by congestive heart disease. While advances have been made in cardiac harness technology, a satisfactory device and method for delivering and positioning the cardiac harness onto a patient's heart has yet to be provided.

Present cardiac harness delivery assemblies use a rigid suction cup to attach at the apex of the heart to hold a delivery device in a desired position relative to the heart. An example of such a rigid suction cup is shown in U.S. patent application Ser. No. 10/704,376, which is hereby incorporated by reference. The rigidness of the suction cup limits its versatility and may present problems with the deployment of a cardiac harness. Complications can arise when the rigid suction cup is not attached directly to the apex of the heart. Further, it may be difficult to maintain a vacuum or suction on the heart tissue of a beating heart with a rigid suction cup. There may also be a problem with the rigid suction cup in that it is not able to conform to the shape of the individual patient's heart.

Therefore, what is needed is a device designed to grasp and hold a beating heart of a patient while a cardiac harness is advanced over the heart. Also, a device is needed that can conform to the various shapes and sizes of a patient's heart and maintain a vacuum or suction on the beating heart. It is also important that the device be designed for use through a minimally invasive access site.

### SUMMARY OF THE INVENTION

The present invention solves the problems associated with the prior art suction cups used to help advance a cardiac harness over the heart. In accordance with the present invention, a suction cup for use with a delivery device for delivering a cardiac harness to the heart, includes a cup body and a connecting aperture connected to the cup body. The cup body is formed of a flexible material, such as silicone rubber, and the cup body has an upper portion and a lower portion, the lower portion defining an opening leading to an inner area of the cup body. In one embodiment, the connecting aperture is formed of a rigid material and is attached to the upper portion of the cup body. In use, the connecting aperture forms a luer connection used to connect the suction cup to a pump member to create a vacuum chamber inside the suction cup. Other connections known in the art may be formed with the connecting aperture instead of a luer connection.

One method of forming a suction cup of the present invention includes placing a rigid connecting aperture in a mold and then casting a pourable silicone rubber around the rigid connecting aperture to form a cup body. The cup body is generally bell shaped, and the lower portion of the cup body can define an opening that is circular, elliptical, or any other shape that is desirable for attaching to the heart. In general, the outside surface of the cup body includes at least one taper, and the lower portion of the cup body flares radially outward. The suction cup is also formed so that a wall of the cup body having a radial width includes a taper. In one embodiment, the radial width of the wall tapers down towards the opening of the cup body on the inside surface of the cup body, thereby increasing the flexibility of the cup body near the opening.

The present invention suction cup has several advantages, including the ability to attach to other areas of the epicardium besides the apex, and the ability to re-center itself onto the apex of the heart when the suction cup is not properly aligned during the initial stages of the delivery procedure. Further, the flexible cup body is able to rotate with respect to the epicardium while maintaining a vacuum pressure inside the cup body. The properties of the silicone rubber allow the suction cup to move along the epicardial surface of a beating heart without injury to the heart and while maintaining a seal and preserving the vacuum attachment to the heart. Also, the flexible suction cup is able to conform to various shapes and sizes of hearts while maintaining a seal along the surface of the heart.

In another embodiment, a suction cup includes a cup body having a wall that is formed of a flexible material. The cup body has a first end and a second end, with the second end defining an opening leading to an inner area defined by the cup body. Preferably, the opening defines a circular opening, however, the opening may be elliptical shaped, ovoid shaped, or any other shape that is desired and compatible with the surface of the heart. An outer surface of the suction cup is bell shaped, however, other shapes such as cone or cylindrical may be used as well. The suction cup of this embodiment also includes a connecting aperture that is connected to the first end of the cup body. There are suction ports disposed around the second end of cup body, and the suction ports are in fluid communication with vacuum lumens that are disposed within the wall of the cup body. The vacuum lumens are disposed along the length of the wall and continue through the connecting aperture to a proximal end of a delivery device, where the vacuum lumens are in fluid communication with a vacuum source, such as a syringe. Any number of suction ports may be disposed at the second end of the cup body, so long as the suction cup has enough suction to hold onto the surface of a beating heart.

In yet another embodiment, the device for holding a heart includes a suctioning strip. The suctioning strip includes a body having a first end and a second end, and a cross-section of the suctioning strip is rectangular, although the cross-section of the suctioning strip may be any shape, including, among others, circular, oval, or square. A connecting aperture 138 may be connected to the first end of the body, although the body of the suctioning strip may not need to be connected to a connecting aperture. The second end of the suctioning strip includes a plurality of suction ports, which are in fluid communication with vacuum lumens disposed within the body of the suctioning strip. The vacuum lumens are disposed along the length of the body and continue through the connecting aperture to a proximal end of a delivery device, where the vacuum lumens are in fluid communication with a vacuum source, such as a syringe. Any number of suction ports may be disposed at the second end of the body, so long as a the suction cup has enough suction to hold onto the surface of a beating heart.

In yet another embodiment, the device for grasping onto a heart includes a plurality of suction tubes, each having a tube with a proximal end and a distal end, and a suction cup attached to the distal end of the tube. An array of suction tubes may be housed within a delivery tube that is advanced through a minimally invasive incision to the heart, and may extend from a distal end of the delivery tube in order to attach to the surface of a beating heart. There is a lumen disposed within each to the tubes, and the lumen is in fluid communication with the suction cups at the distal end and in fluid communication with a vacuum source, such as a syringe, at the proximal end. The tubes may be formed of a flexible material or a semi-rigid material.

### BRIEF DESCRIPTION OF THE DRAWINGS

FIG. 1 depicts an elevational view of a suction cup of the present invention.

FIG. 2 depicts a cross-sectional view of the suction cup shown in FIG. 1.

FIG. 3 is a side elevational view of one embodiment of a delivery device including a suction cup, which is configured to securely hold the heart relative to the delivery device during advancement of the cardiac harness over the heart.

FIG. 4 is a side elevational view of the delivery device of FIG. 3 with the cardiac harness in a partially advanced position.

FIG. 5 is a side elevational view of the delivery device of FIG. 3 with the cardiac harness fully advanced over the heart.

FIG. 6 depicts the suction cup of the present invention attached to the side of the apex of the heart.

FIG. 7 depicts the suction cup of FIG. 6 being tensioned in a direction away from the heart, and the suction cup sliding towards the apex of the heart.

FIG. 8 depicts the suction cup of FIGS. 6 and 7 positioned on the apex of the heart, which is tensioned and slightly elongated.

FIG. 9 depicts an elevational view of another embodiment of a suction cup.

FIG. 10 depicts a cross-sectional view of the suction cup shown in FIG. 9.

FIG. 11 depicts a bottom planar view of the suction cup shown in FIG. 9.

FIG. 12 depicts a cross-sectional view of a suctioning strip.

FIG. 13 depicts a bottom planar view of the suctioning strip shown in FIG. 12.

FIG. 14 depicts another embodiment including an array of suctioning tubes.

FIG. 15 depicts a cross-sectional view of a suctioning tube from FIG. 14.

### DETAILED DESCRIPTION OF THE PREFERRED EMBODIMENTS

One embodiment is directed to a suction cup for use with a delivery device for delivering a cardiac harness to a beating heart. The suction cup is associated with the delivery device and is used to attach at the apex of the heart to maintain the position of the delivery device with respect to the heart. Once the suction cup is placed on the heart, air is withdrawn from an inner area of the suction cup to create a vacuum, thereby permitting the suction cup to securely hold the beating heart of a patient. The suction cup can then be pulled in a proximal direction away from the heart to counteract the deployment forces required to deliver and advance a cardiac harness over the heart.

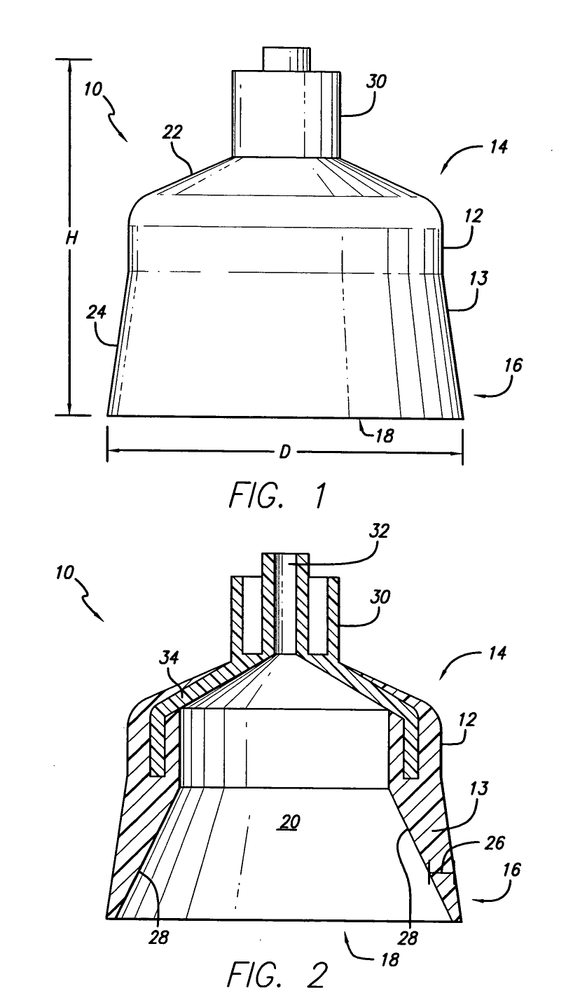

Referring to FIGS. 1 and 2, one embodiment of a suction cup 10 includes a cup body 12 having a wall 13. The cup body is formed of a flexible material, preferably silicone rubber. However, any soft and playable material may be used, including any thermoplastic elastomers or rubbers, such as kraton, santoprene, urethanes, silicones or natural rubbers. The cup body has an upper portion 14 and a lower portion 16, with the lower portion defining an opening 18 leading to an inner area 20 defined by the cup body. The lower portion preferably defines a circular opening, however, the lower portion may define an elliptical opening or any other shape that is desired and compatible with the surface of the heart.

As shown in FIGS. 1 and 2, an outer surface of the cup body 12 is generally bell shaped and includes a taper 22 on the upper portion 14 of the cup body, and a taper 24 on the lower portion 16 of the cup body. The outer surface shape of the cup body is such that a diameter of the cup body taken along a cross-section at the upper portion 14 is less than the diameter taken along a cross-section at the lower portion 16. The taper on the lower portion of the cup body causes the lower portion of the cup body to flare radially outward. In this embodiment, the shape of the cup body provides an advantage for delivering the cardiac harness over the heart (discussed below), because the tapers on the outer surface of the suction cup provide a smooth transition for the cardiac harness as it is pushed over the suction cup and onto at least a portion of the heart. As the cardiac harness is pushed from the delivery device, the cardiac harness slides over the tapered surface of the suction cup 10 and onto the surface of the heart.

The suction cup 10 is also formed so that the wall 13 has a radial width 26 that includes a taper 28 on the inner surface of the cup body 12. In one embodiment, the taper of the wall decreases in thickness towards the opening of the cup body, so that the radial width of the wall is thinnest at the lower portion 16 of the cup body near the opening 18. This design gives the lower portion of the suction cup increased flexibility, allowing the suction cup to more easily form a seal with the surface of the beating heart. Another feature of this design is that the suction cup is able to conform to various shapes and sizes of different hearts.

The suction cup 10 also includes a connecting aperture 30 formed of a rigid material, such as any hardened plastic, that is attached to the upper portion 14 of the cup body 12. This rigid portion may be formed of any rigid thermoplastic, including polycarb, polypropylene, ABS, polyethylene, or vinyl. A channel 32 is formed within the connecting aperture, and the channel is in fluid connection with the inner area 20 of the cup body. As shown in FIG. 2, one embodiment of the connecting aperture includes a radial structure 34 that connects to the upper portion of the cup body. The radial structure allows the connecting aperture to be attached to the flexible cup body and it provides a stiffness to the upper portion of the cup body to help maintain a vacuum chamber over the heart surface. Also, the rigid radial structure of the connecting aperture forces the cardiac harness to expand when being delivered over the suction cup and onto the surface of the heart.

In one embodiment, the connecting aperture 30 provides a luer connection used to connect the suction cup 10 to the shaft of a delivery system as well as to a suction tubing and suction valve assembly, which are used to create a vacuum within the suction cup. Delivery devices that are configured to locate and grasp a heart with the use of a suction cup are discussed in Applicants' co-pending applications entitled “Cardiac Harness Delivery Device and Method” U.S. Ser. No. 10/715,150 filed Nov. 17, 2003, and “Cardiac Harness For Treating Congestive Heart Failure And For Defibrillating And Pacing” U.S. Ser. No. 10/704,376 filed Nov. 7, 2003, the entirety of each is hereby expressly incorporated by reference.

In one embodiment, the connecting aperture 30 is formed by cutting off the bottom end of a syringe. A syringe can be used because it is formed of a rigid material and is able to provide a luer connection between the suction cup 10 and the shaft of the delivery system. In other embodiments, the connecting aperture is separately molded using any rigid polymer. The rigid nature of the connecting aperture allows the suction cup to maintain a vacuum chamber over the epicardium, thereby maximizing the holding power of the suction cup for a given vacuum pressure.

A method of manufacturing the suction cup 10 for attaching to the surface of the heart includes placing the rigid connecting aperture 30 in a mold and then casting a pourable silicone around the rigid connecting aperture to form the cup body 12. Preferably, the cup body is formed so that its outer surface is tapered, and the lower portion 16 of the cup body forms a circular opening, although the opening may be formed into other shapes including an elliptical shape. The flexible cup body of the suction cup, formed of silicone rubber in this embodiment, provides a resilient seal to prevent the loss of vacuum pressure inside the suction cup during manipulation of the delivery device. Further, the flexible cup body allows the suction cup to attach to a variety of heart shapes, contours, and sizes.

It is possible to manufacture the suction cup 10 in a range of sizes and shapes. Specifically, the diameter of the cup body 12 at the lower portion 16 forming the opening 18, designated in FIG. 1 by line D, can range from about 0.1 inches to about 2.0 inches, and is preferably about 1.2 inches. Further, the height of the suction cup designated in FIG. 1 as line H, can range from about 0.375 inches to about 2.0 inches, and is preferably about 1.31 inches. The height of the cup body 12 alone can range from about 0.25 inches to about 1.0 inches. The radial width of the wall 13 can range anywhere from about 0.125 inches to about 0.25 inches at the upper portion, and can range from about 0.01 inches to about 0.25 inches at the lower portion including the taper 28. Further, the durometer of the flexible cup body can range from about 20 shore OO to about 60 shore A.

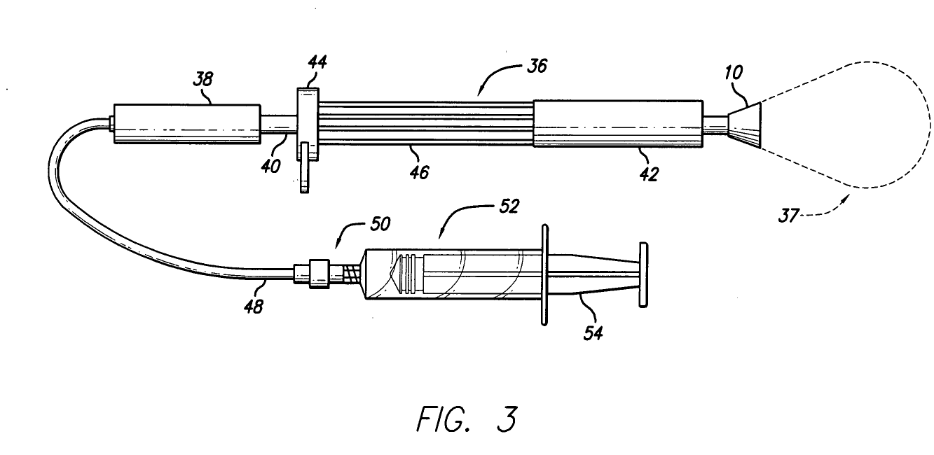

A method for delivering a cardiac harness assembly to a beating heart through a minimally invasive access site is disclosed in U.S. Ser. No. 10/715,150 and in U.S. Ser. No. 10/704,376, which have already been incorporated by reference above. In one embodiment, a minimally invasive access site is created on the torso of the patient near the apex of the heart, and a delivery device 36 including a suction cup 10 is inserted there through. With reference to FIG. 3, the delivery device of this embodiment includes a handle 38 affixed to the proximal end of an elongate shaft 40. Preferably, a housing 42 is affixed to a distal end of the elongate shaft. A control assembly 44 and a plurality of push rods 46 are also included, and are axially slidable along the elongate shaft. The cardiac harness (not shown) is releasably supported on the distal end portions of the push rods in a compacted configuration within the housing.

To hold the delivery device 36 in a desired position relative to the heart 37, the connecting aperture 30 of the suction cup 10 is attached to a distal end of the elongated shaft 40, and a tube 48 extending through the elongated shaft is connected to the connecting aperture of the suction cup. A distal end of the tube is in fluid connection with the channel of the connecting aperture, and a proximal end of the tube includes a connector 50 that allows connection of the tube to a pump member 52, such as a syringe or other source of vacuum. It is preferred that the syringe is connected to the tube with a plunger 54 of the syringe being in a compressed position as shown in FIG. 3. Once the suction cup is in contact with the surface of the heart, the plunger is retracted to create a vacuum condition within the tube, and thus within the inner area of the suction cup. Due to the vacuum condition, the suction cup grasps the surface of the heart such that the heart is held in a desired position relative to the delivery device.

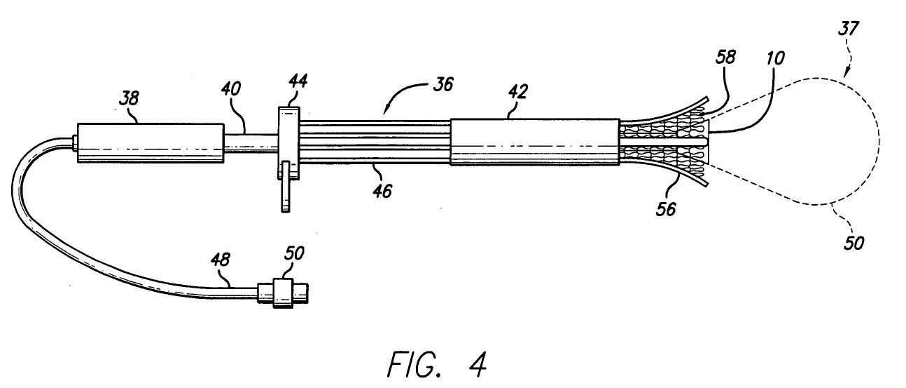

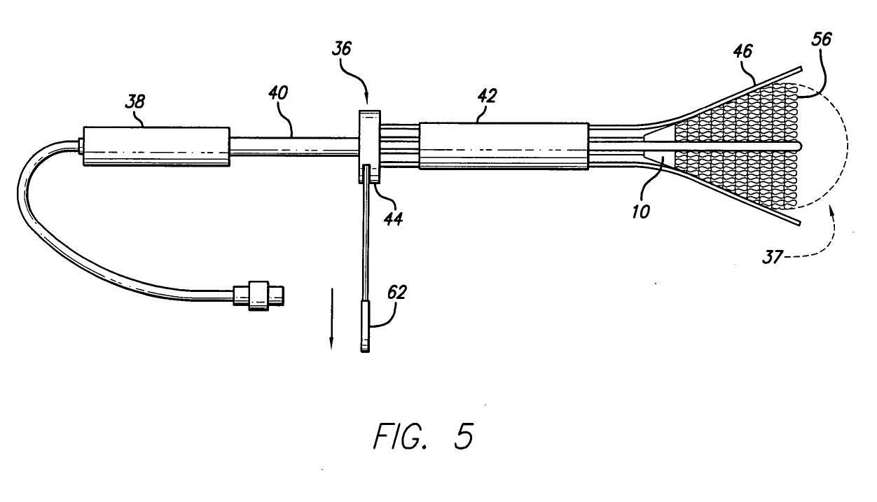

Once the delivery device 31 has been secured to the apex of the heart 37, the cardiac harness, designated 56, is deployed from the housing 42 and positioned over the heart as shown in FIGS. 4-5. FIG. 4 shows the cardiac harness being advanced from its compacted configuration within the housing onto the heart in a direction from an apex portion 58 to a base portion 60 of the heart. As shown, the harness is pushed over the tapered surface of the suction cup 10 and stretched elastically to fit over the heart. It should be understood that a substantially non-elastic harness embodiment can also be delivered by this device and method.

With next reference to FIG. 5, the control assembly 44 continues to be advanced until the cardiac harness 56 is properly positioned, and then a release member 62 is pulled away from the control assembly 44. Accordingly, the cardiac harness is released from the plurality of push rods 46. After the cardiac harness is in place over at least a portion of the heart 37, the vacuum pressure is released, allowing the suction cup 10 to be removed from the heart and the delivery device 36 to be removed from the patient.

Although one embodiment of a method to deliver a cardiac harness to a heart is discussed above, the suction cup of the present invention can be used with any delivery device used to deliver a cardiac harness to the heart. Further, other suitable pump devices may be used in place of a syringe to create a vacuum within the suction cup, as will be appreciated by one of ordinary skill in the art.

The suction cup 10 of the present invention has performed well in animal studies for attaching to the epicardial apex of the heart. These studies have revealed several advantages of the suction cup of the present invention. Specifically, the flexible cup body 12 allows the suction cup to attached onto other areas of the heart besides the apex, and the suction cup of the present invention can also re-center itself by sliding on the epicardial surface to the apex of the heart when the suction cup is not properly aligned on the heart during the initial stages of the delivery procedure. Further, the flexible cup body is able to rotate with respect to the epicardial surface while maintaining the vacuum attachment to the heart. These advantages were noticed during the animal studies when the suction cup was successfully attached to the free walls of the ventricles. It was also noticed that when the suction cup was initially attached to the free wall of the ventricle instead of the apex, tension could be applied to the delivery device causing the suction to slide along the epicardial surface until the suction cup was re-centered onto the apex of the heart.

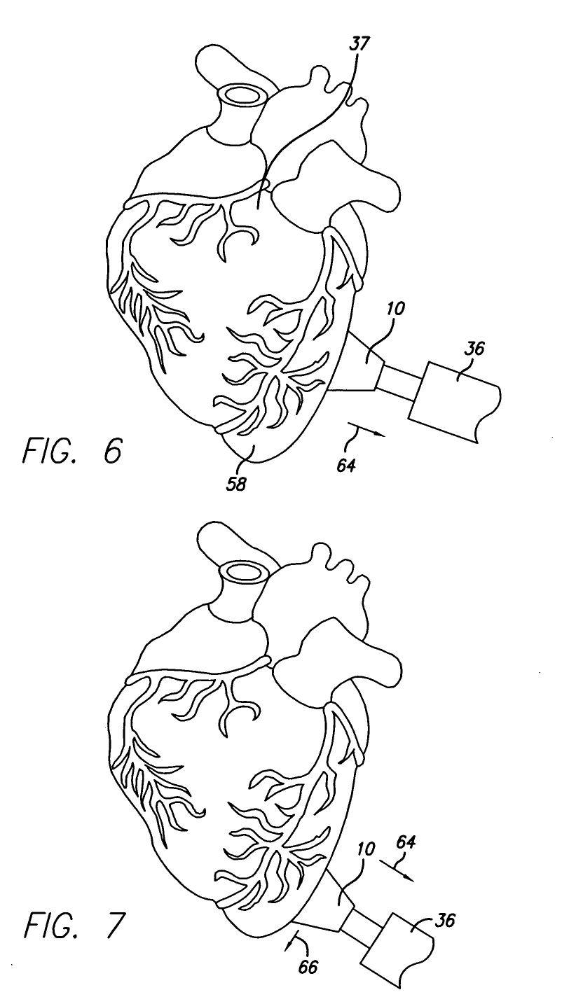

These advantages were notice in other animal studies as well. During one study, the access incision mistakenly was not placed directly over the apex of the heart, and thus the suction cup had to be attached to the side of the apex instead of at the very tip of the apex. Continuing with the procedure, the delivery system including the suction cup was tensioned to prepare for deployment of the cardiac harness, causing the suction cup to slide onto the tip of the apex into a more favorable pre-deployment position. This feature of the present invention is shown in FIGS. 6 through 8. In FIG. 6, the suction cup 10 is shown to be initially attached to the heart 37 on the side of an apex 58 of the heart. By pulling back on the delivery device 36 including the suction cup in a proximal direction away from the heart as indicated by arrow 64, the suction cup begins sliding on the epicardial surface of the heart. FIG. 7 shows the suction cup sliding in a direction toward the apex of the heart, indicated by arrow 66, as the suction cup is tensioned in the proximal direction. The suction cup of the present invention will re-center itself at the apex of the heart as shown in FIG. 8.

As the delivery system is slightly pulled back in a proximal direction, the heart is tensioned and even elongates slightly as shown in FIG. 8. The suction cup pulls on the heart and since the great vessels (aorta, etc.) are at the base of the heart and hold it from that end, the heart will tension under the pulling force of the suction cup and delivery system at the opposite, apex end of the heart. By tensioning the heart, the cardiac harness slides more easily over the heart because there is a resistance or push/pull relationship. As the heart is tensioned by pulling back on the suction cup and the delivery system, the cardiac harness is pushed forward distally past the suction cup and over and onto the heart.

The ability of the present suction cup to re-center itself onto the apex of the heart is due to the smooth inner surface of the cup body and the lubricity of the silicone polymer that serves as the seal. The silicone rubber material becomes very slippery when it is in contact with the moist epicardial tissue of the heart, and this allows the suction cup to move and rotate along the epicardium while still maintaining a seal and preserving the vacuum attachment to the heart. The suction cup does not increase the sliding friction against the epicardium because there are no internal lips, edges, or dissimilar materials on the smooth inner surface of the cup body.

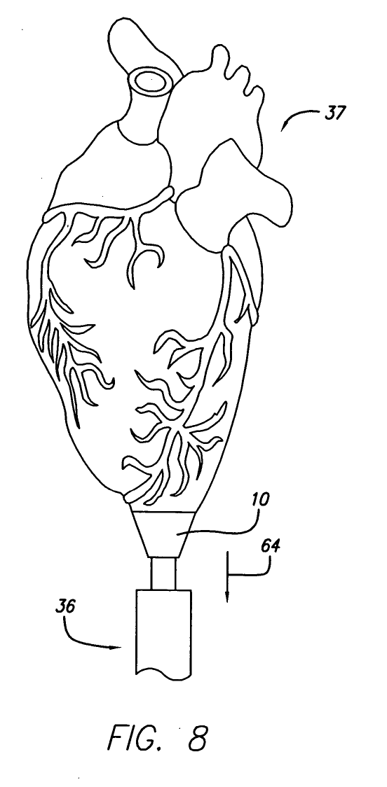

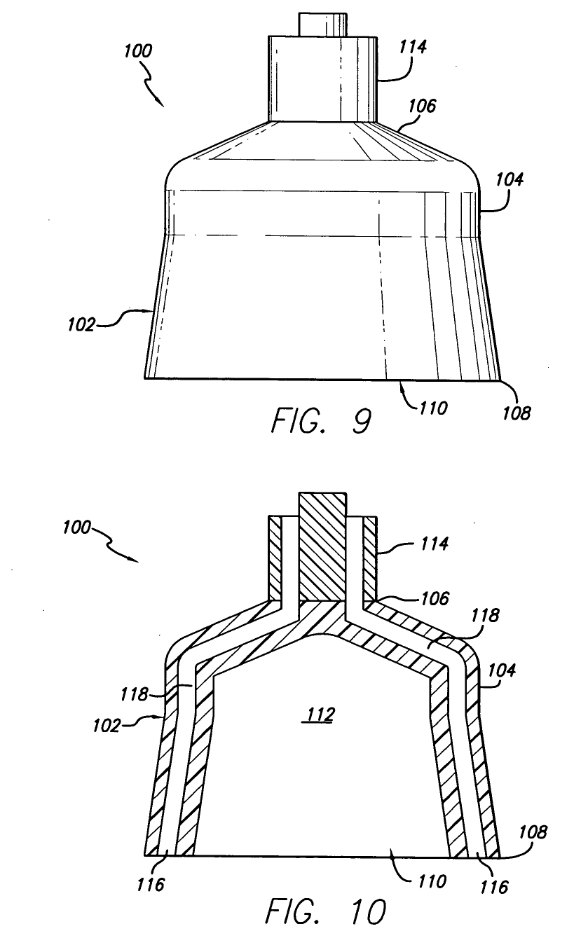

Another embodiment of a suction cup 100 is shown in FIGS. 9 through 11. As shown in FIG. 9, the suction cup includes a cup body 102 having a wall 104 that is formed of a flexible material. The cup body can be any elastomer or rubber, such as kraton, santoprene, urethanes, silicones, or natural rubbers. The cup body has a first end 106 and a second end 108, with the second end defining an opening 110 leading to an inner area 112 defined by the cup body. The opening preferably defines a circular opening, however, the opening may be elliptical shaped, ovoid shaped, or any other shape that is desired and compatible with the surface of the heart. An outer surface of the suction cup is bell shaped similar to the suction cup 10 shown in FIG. 1, however, other shapes such as cone or cylindrical may be used as well. The suction cup also includes a connecting aperture 114 that is connected to the first end of the cup body. It is preferable that the connecting aperture be formed of a rigid material, such as a hardened plastic to provide a stiffness to the upper portion of the cup body and to help maintain a suction on the heart surface. The rigid portion may be made from any rigid thermoplastic, including polycarb, polypropylene, ABS, polyethylene, or vinyl. Also, the rigid structure of the connecting aperture forces the cardiac harness to expand when being delivered over the suction cup and onto the surface of the heart.

FIG. 10 depicts a cross-sectional view of the suction cup 100 shown in FIG. 9. In one embodiment, suction ports 116 are disposed around the cup body 102 at the second end 108 of the cup body, and the suction ports are in fluid communication with vacuum lumens 118 that are disposed within the wall 104 of the cup body. The vacuum lumens are disposed along the length of the wall and continue through the connecting aperture 114 to a proximal end of a delivery device, where the vacuum lumens are in fluid communication with a vacuum source, such as a syringe. Several suction ports are disposed around the opening 110 of the cup body, as shown in FIG. 11. Any number of suction ports may be disposed at the second end of the cup body, so long as the force created at the suction ports is great enough to hold onto the surface of a beating heart.

The cup body 102 and the connecting aperture 114 can be manufactured similar to the suction cup 10 discussed above. An optimal combination of materials for the flexible body and rigid aperture would be a rigid plastic and a flexible elastomer that bond to each other when manufactured, to decrease the risk of the flexible portion becoming detached. The flexible cup body allows the suction cup 100 to attach to a variety of heart shapes, contours, and sizes. It is possible to manufacture the suction cup 100 in a range of sizes and shapes. Specifically, the diameter of the cup body at the second end 108 can range from about 0.1 inches to about 2.0 inches, and is preferably about 1.2 inches. Further, the height of the cup body from the first end 106 to the second end 108 can range from about 0.25 inches to about 1.0 inches, and the height of the connecting aperture may range up to 1.0 inches. The radial width of the wall 104 can range from about 0.125 inches to about 0.25 inches. In one embodiment, the radial width of the wall is constant from the first end to the second end, however, the radial width of the wall may vary from the first end to the second end and may even include a taper. The size of the suction ports 116 may range from microscopic sizes up to any size that will fit with the space constrains of the wall. In one embodiment, the diameter of the suction ports may range up to about 0.25 inches. The suction ports of microscopic sizes may be formed using lasers or chemical etching. Further, the durometer of the flexible cup body can range from about 20 shore OO to about 60 shore A.

This embodiment of the suction cup 100 would be used as described above with the first embodiment of suction cup 10, except that when a vacuum is created within the individual vacuum lumens 118, the suction ports 116 would suction onto the surface of the beating heart. The flexible cup body 102 would also have similar features and advantages of the flexible cup body 12 of the first embodiment.

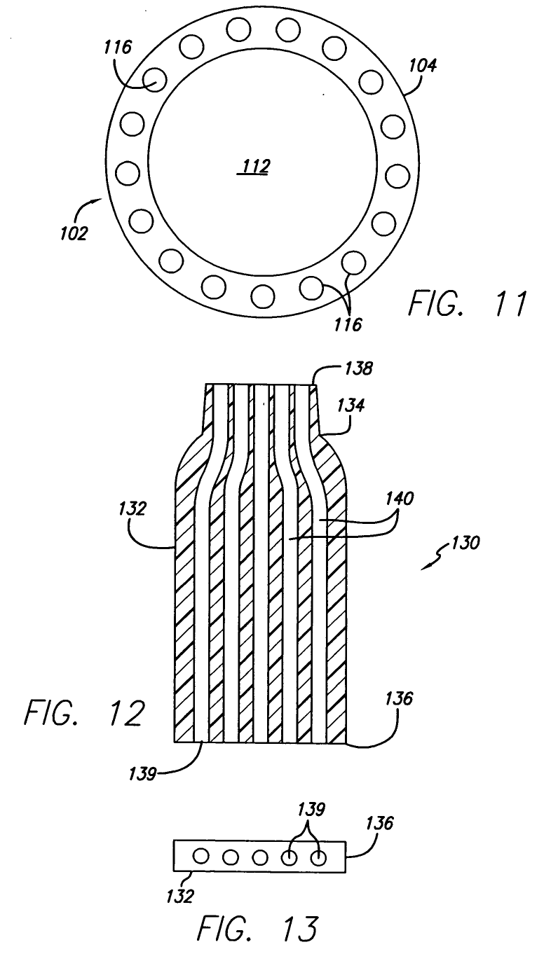

FIG. 12 depicts an embodiment of a suctioning strip 130 that may be used in place of a suction cup to suction onto the surface of a beating heart. The suctioning strip includes a body 132 having a first end 134 and a second end 136, and a cross-section of the suctioning strip is rectangular, although it may have any shape, such as circular, oval, or square. A connecting aperture 138 is connected to the first end of the body. The second end of the suctioning strip includes a plurality of suction ports 139, which are best shown in FIG. 13. Suction ports are in fluid communication with vacuum lumens 140 disposed within the body of the suctioning strip. The vacuum lumens are disposed along the length of the body and continue through the connecting aperture to a proximal end of a delivery device, where the vacuum lumens are in fluid communication with a vacuum source, such as a syringe. Any number of suction ports may be disposed at the second end of the body, so long as the suctioning strip creates enough suction to hold onto the surface of a beating heart.

In this embodiment, the body 132 of the suctioning strip 130 may be made with a flexible material or a rigid material. The same elastomers, rubbers, or rigid thermoplastics disclosed above could be used in manufacturing the suctioning strip. It is also possible that the connecting aperture 138 and the body 132 may be formed of the same material. The structure of the suctioning strip's body should provide enough stiffness to the device without the need for a rigid connecting aperture. In one embodiment the second end 136 of the body includes a contact surface 142 that may be flat along its edge as shown in FIG. 12, or the contact surface may include a curve to better follow the contour of the heart. A length of the contact surface may range from about 0.125 inches to about 2.0 inches, with a width of about 0.125 inches to about 2.0 inches. The size of the suction ports 134 may range from a microscopic size up to any size that will fit within the space constrains of the contact surface. In one embodiment, the diameter of the suction ports may range up to about 0.25 inches. The suction ports of microscopic sizes may be formed using lasers or chemical etching. Still in another embodiment, the cross-section of the suctioning strip may be square, and the suctioning strip may include only one suction port having a diameter of about 2.0 inches.

In use, the contact surface 142 of the suctioning strip 130 would come into contact with the surface of the beating heart, and a vacuum would then be created within the vacuum lumens 140 creating a suctioning force at the suction ports to grip the heart. Any restraint for the heart including a cardiac harness could then be advanced over the suctioning strip and onto the surface of the beating heart.

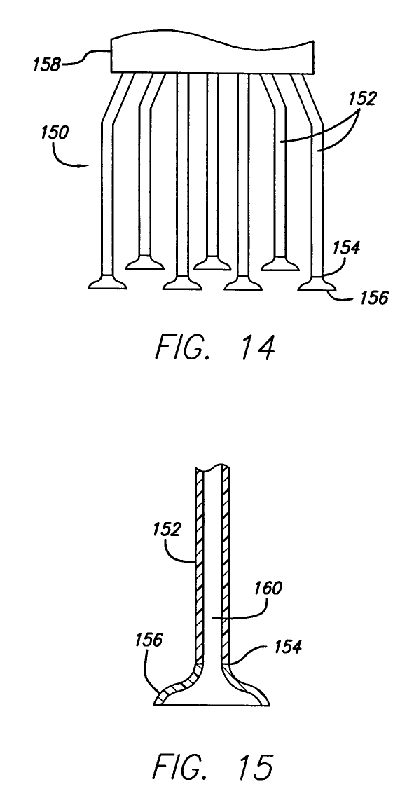

Another embodiment is shown in FIGS. 14 and 15, and includes a plurality of suction tubes 150 having a tube 152 with a proximal end (not shown) and a distal end 154, and a suction cup 156 attached to the distal end of the tube. An array of suction tubes may be housed within a delivery tube 158 that is advanced through a minimally invasive incision to the heart, and may extend from a distal end of the delivery tube in order to attach to the surface of a beating heart. A lumen 160 is disposed within each of the tubes, and the lumen is in fluid communication with the suction cups at the distal end and in fluid communication with a vacuum source, such as a syringe, at the proximal end.

The tubes 152 may be formed of a flexible material or a semi-rigid material. Preferably the tubes have enough stiffness to extend from the delivery tube 158 and come into contact with the surface of the heart. The suction cups 156 can be made from any flexible material, such as thermoplastic elastomers or rubbers. The diameter of the suction cups 156 can range from about 0.100 inches to about 0.750 inches.

In use, the distal ends 154 of the suction tubes 150 are extended from the distal end of the delivery tube 158, bringing the suction cups 156 in contact with the surface of the patient's beating heart. In another embodiment, the suction tubes are delivered directly to the patient's heart without the use of a delivery device. Once the suction cups are on the surface of the heart, a vacuum is created within each vacuum lumen, causing the suction cups to each grasp onto the surface of the heart. The suction tubes can the be pulled in a proximal direction, thereby tensioning the heart, and a cardiac harness can then be advanced over the suction tubes and onto the surface of the beating heart. One advantage of using several individual suction tubes is that they will conform to any heart regardless of its shape and size.

Although the present invention has been described in terms of certain preferred embodiments, other embodiments that are apparent to those of ordinary skill in the art are also within the scope of the invention. Accordingly, the scope of the invention is intended to be defined only by reference to the appended claims. While the dimensions and types of materials described herein are intended to define the parameters of the invention, they are by no means limiting and are exemplary embodiments.

We claim:

1. A suction cup for use with a delivery device for delivering a cardiac harness to a heart, comprising:
a cup body formed of a flexible material, the cup body having an upper portion and a lower portion, wherein the lower portion defines an opening leading to an inner area of the cup body; and
a connecting aperture formed of a rigid material being attached to the upper portion of the cup body.

2. The suction cup of claim 1, wherein the flexible material of the cup body is selected from the group consisting of kraton, santoprene, urethane, silicone, and natural rubber.

3. The suction cup of claim 1, wherein the cup body is formed of a material including silicone.

4. The suction cup of claim 1, wherein the lower portion defines a circular opening.

5. The suction cup of claim 1, wherein the lower portion defines an elliptical opening.

6. The suction cup of claim 1, wherein the connecting aperture includes a channel that is in fluid connection with the inner area of the cup body.

7. The suction cup of claim 1, wherein the connecting aperture is connected to a means for creating a vacuum in the inner area of the cup body.

8. The suction cup of claim 7, wherein the means for creating a vacuum in the inner area of the cup body includes a syringe.

9. The suction cup of claim 1, wherein the cup body includes a wall having a radial width that includes a taper.

10. The suction cup of claim 9, wherein the connecting aperture includes a radial structure attached to the upper portion of the cup body, and the upper portion of the cup body is rigid.

11. The suction cup of claim 1, wherein the cup body has a diameter at the lower portion defining the opening between about 0.5 inches and about 2.0 inches.

12. The suction cup of claim 1, wherein the flexible material has a durometer of about 20 shore OO to about 60 shore A.

13. A method of manufacturing a suction cup for attaching to the apex of a heart, comprising:
providing a rigid connecting aperture having a channel; and
placing the rigid connecting aperture in a mold and casting a pourable silicone around the rigid connecting aperture to form a cup body having an upper portion and a lower portion, wherein the lower portion defines an opening leading to an inner area of the cup body.

14. The method of claim 13, further comprising providing a tubing to be in fluid connection with the channel of the rigid connecting aperture, wherein the tubing can be connected to a pump member for creating a vacuum in the inner area of the cup body.

15. The method of claim 13, wherein the lower portion of the cup body defines a circular opening.

16. The method of claim 13, wherein the lower portion of the cup body defines an elliptical opening.

17. The method of claim 13, wherein the silicone forming the cup body has a durometer of about 20 shore OO to about 60 shore A.

18. The method of claim 13, wherein the cup body has a tapered outer surface.

19. The method of claim 13, wherein the cup body includes a wall having a radial width that includes a taper.

20. A method for delivering a cardiac harness assembly to the heart, comprising:
creating a minimally invasive access site;
inserting a delivery device for carrying a cardiac harness through the minimally invasive access site, wherein the delivery device includes a suction cup having a cup body formed of a flexible polymer;
attaching the suction cup to the heart; and
delivering the cardiac harness to the heart.

21. The method of claim 20, further comprising re-centering the suction cup onto the apex of heart.

22. The method of claim 20, wherein attaching the suction cup to the heart, the suction cup is attached on the apex of the heart.

23. The method of claim 20, wherein attaching the suction cup to the heart, the suction cup is attached to any portion of the ventricle walls of the heart.

24. The method of claim 20, further comprising tensioning the delivery device and suction cup attached to the heart in a proximal direction elongating the heart.

25. The method of claim 24, wherein delivering the cardiac harness to the heart, pushing the cardiac harness over the suction cup and the elongated heart.

26. A method for delivering a cardiac harness to a beating heart, comprising:
attaching a suction cup to the surface of the beating heart, wherein the suction cup includes a body formed of a flexible polymer; and
advancing a cardiac harness over the suction cup and onto the beating heart.

27. The method of claim 26, further comprising creating a minimally invasive access site, wherein the suction cup is attached to the surface of the beating heart through the minimally invasive access site.

28. The method of claim 26, further comprising tensioning the suction cup attached to the beating heart in a proximal direction elongating the heart.
